(19) United States
(12) Patent Application Publication
(10) Pub. No.: US 2010/0331403 A1
(43) Pub. Date: Dec. 30, 2010

(54) COMPOUNDS AND METHODS FOR THE TREATMENT OF CANCER

(76) Inventors: John C. Amedio, JR., Franklin, MA (US); Frank Walter Waligora, Haverhill, MA (US)

Correspondence Address: ROPES & GRAY LLP;IPRM - Floor 43, PRUDENTIAL TOWER, 800 BOYLSTON STREET, BOSTON, MA, 02199-3600, US

(21) Appl. No.: 12/744,655

(22) PCT Filed: Dec. 12, 2008
(86) PCT No.: PCT/US08/13620

§ 371 Date: Sep. 7, 2010

Related U.S. Application Data
(60) Provisional application No. 61/007,371, filed on Dec. 12, 2007.

(51) Int. Cl. A61K 31/285; C07F 9/72; A61P 35/00; G01N 30/00; B01D 59/44; G01R 33/44

(52) U.S. Cl. 514/504; 556/70; 556/71; 73/61.52; 250/282; 324/310

(57) ABSTRACT

The present invention provides methods of synthesizing organic arsenicals. Many of these compounds have potent in vitro cytotoxic activity against numerous human tumor cell lines, both of solid and hematological origin, as well as against malignant blood cells from patients with leukemia.

### CROSS-REFERENCE TO RELATED APPLICATIONS

This application claims the benefit of U.S. Provisional Application Ser. No. 61/007,371, filed Dec. 12, 2007, the specification of which is incorporated herein in its entirety.

### BACKGROUND OF THE INVENTION

S-dimethylarsinoglutathione (SGLU-1) is an organic arsenical currently manufactured for treatment of cancer. Methods for the synthesis of SGLU-1 have, to date, been two-step syntheses where the first step involved the reduction of cacodylic acid with hypophosphorus acid. Unfortunately, use of hypophosphorus acid produces phosphine gas as a side product, which can be hazardous in large quantities. The second step of the synthesis requires use of pyridine as a base, which is difficult to remove completely from the final product. Additionally, pyridine's high boiling point and affinity to the drug substance increased the time needed to dry SGLU-1. What is needed is a method for the synthesis of SGLU-1 that provides a safe and efficient method for large-scale production. Additionally, a method for the synthesis of SGLU-1 with higher purity is needed.

### SUMMARY OF THE INVENTION

One aspect of the invention relates to a method for the synthesis of a compound of formula (I)

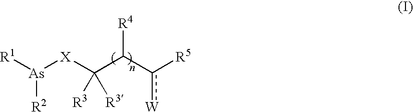

wherein

X is S or Se, preferably S;

W is O, S, or (R)(R), where each occurrence of R is independently H or a C₁₋₂alkyl, preferably O or (R)(R);

n is 0 or 1, preferably 1;

R¹ and R² are independently C₁₋₂₀alkyl, preferably R¹ and R² are independently selected from methyl, ethyl, propyl, and isopropyl;

R³ is —H or C₀₋₆alkyl-COOR⁶;

R^{3′} is H, amino, cyano, halogen, aryl, aralkyl, heteroaryl, heteroaralkyl, carboxyl, C₁₋₁₀alkyl, C₁₋₁₀alkenyl, or C₁₋₁₀alkynyl, preferably H;

R⁴ is —OH, —H, —CH₃, —OC(O)C₁₋₁₀aralkyl, —OC(O)C₁₋₁₀alkyl, —OC(O)aryl, or a glutamine;

R⁵ is —OH, cyano, C₁₋₁₀alkoxy, amino, O-aralkyl, —OC(O)C₁₋₁₀aralkyl, —OC(O)C₁₋₁₀alkyl, —OC(O)aryl, or a glycine substituent; and

R⁶ is H or C₁₋₁₀alkyl, preferably H,

comprising reacting a compound having a structure of formula (II)

(R¹)(R²)AsCl (II)

with a compound having a structure of formula (III)

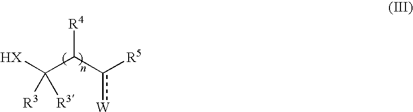

in an aqueous or alcoholic solvent in the absence of pyridine to provide a compound of formula (I).

Another aspect of the invention relates to a method for purifying a compound of formula (I), e.g., following performing the above method, comprising

(a) adding an alcoholic and/or polar aprotic solvent, miscible with both ethanol and water that decreases the solubility of the compound of formula (I), preferably a dialkyl ketone such as acetone, to the reaction mixture, e.g., while agitating; and

(b) filtering the resulting slurry.

In certain embodiments, the method of purification further comprises

(a) preparing a solution of the compound of formula (I) in water;

(b) filtering the solution;

(c) reducing the amount of water (e.g., under reduced pressure and/or by azeotropic distillation);

(d) adding a water miscible, polar aprotic solvent that decreases the solubility of the compound (e.g., acetone, methyl isopropyl ketone, methyl ethyl ketone, or tetrahydrofuran), preferably a dialkyl ketone such as acetone, to form a slurry; and

(e) filtering the resulting slurry.

One aspect of the invention is a method for determining or monitoring the purity of SGLU-1. More particularly, such an assay is for determining or monitoring the presence of organic arsenical impurities resulting from the manufacture of SGLU-L Such methods may include, but are not limited to, mass spectrometry, high pressure liquid chromatography (HPLC), and nuclear magnetic resonance (NMR), and combinations of such techniques, such as liquid chromatography-mass spectrometry (LC-MS).

One aspect of the invention relates to a method for determining or monitoring the presence and/or the amount of a compound of Formula VIII

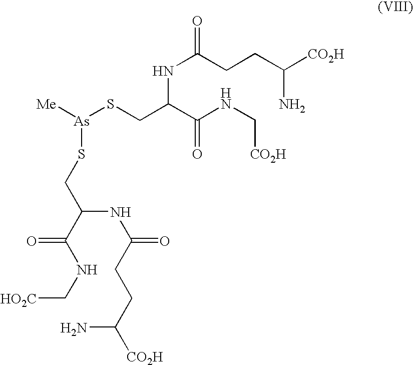

or a salt thereof in a sample of SGLU-1.

One aspect of the invention relates to a method of manufacturing a pharmaceutical formulation of SGLU-1, comprising determining the amount of a compound of Formula VIII or salt thereof that is present in a sample of SGLU-1 and if a compound of Formula VIII or salt thereof is present in an amount less than about 5% (w/w), adding a pharmaceutically acceptable diluent, carrier, or excipient.

### DETAILED DESCRIPTION OF THE INVENTION

One aspect of the invention relates to a method for the synthesis of a compound of formula (I)

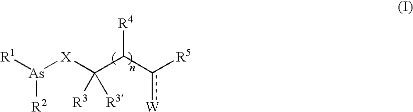

wherein

X is S or Se, preferably S;

W is O, S, or (R)(R), where each occurrence of R is independently H or a C₁₋₂alkyl, preferably O or (R)(R);

n is 0 or 1, preferably 1;

R¹ and R² are independently C₁₋₂₀alkyl, preferably R¹ and R² are independently selected from methyl, ethyl, propyl, and isopropyl;

R³ is —H or C₀₋₆alkyl-COOR⁶;

R^{3′} is H, amino, cyano, halogen, aryl, aralkyl, heteroaryl, heteroaralkyl, carboxyl, C₁₋₁₀alkyl C₁₋₁₀alkenyl, or C₁₋₁₀alkynyl, preferably H;

R⁴ is —OH, —H, —CH₃, —OC(O)C₁₋₁₀aralkyl, —OC(O)C₁₋₁₀alkyl, —OC(O)aryl, or a glutamine;

R⁵ is —OH, cyano, C₁₋₁₀alkoxy, amino, O-aralkyl, —OC(O)C₁₋₁₀aralkyl, —OC(O)C₁₋₁₀alkyl, —OC(O)aryl, or a glycine substituent; and

R⁶ is H or C₁₋₁₀alkyl, preferably H,

comprising reacting a compound having a structure of formula (II)

(R¹)(R²)AsCl (II)

with a compound having a structure of formula (III)

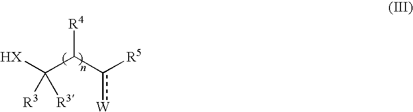

in an aqueous or alcoholic solvent and in the absence of pyridine to provide a compound of formula (I).

In certain embodiments, the reaction is done in the presence of a non-aromatic amine base. In certain such embodiments, the non-aromatic amine base is selected from triethylamine and diisopropylethylamine, preferably triethylamine.

In certain embodiments, the mole ratio of the compound of formula (II) to the non-aromatic amine base is between about 0.5:1 to about 1.5:1. In certain such embodiments, the mole ration is about 0.7:1 to about 1.3:1, or even about 1:1 to about 1.1:1. In certain such embodiments, the mole ratio is about 1:1 or even about 1.1:1.

In certain embodiments, the mole ratio of the non-aromatic amine base to the compound of formula (III) is between about 1:1 to about 2:1. In certain such embodiments, the mole ratio is between about 1.1:1 to about 1.5:1, or even about 1:1 to about 1.3:1. In certain such embodiments, the mole ratio is about 1.1:1, 1.2:1, or even about 1.3:1.

In certain preferred such embodiments, the solvent system comprises water and ethanol. In certain embodiments, the ratio of water to ethanol (v/v) is between about 4:1 and 1:4, preferably between about 2:1 and about 1:2. In certain preferred such embodiments, the ratio of water to ethanol (v/v) is about 1:1.

In certain embodiments, such a method is performed such that the yield of the compound of formula (I) is at least about 50%, about 60%, about 75%, about 80%, about 85%, about 90%, about 95% or even quantitative.

In certain embodiments, the compound of formula (I) is at least about 97% pure as measured by HPLC and is free of pyridine. In certain preferred embodiments, the compound is at least about 99.5% pure.

In certain embodiments, the method further comprises

(a) adding an alcoholic and/or polar aprotic solvent, miscible with both ethanol and water that decreases the solubility of the compound of formula (I), preferably a dialkyl ketone such as acetone, to the reaction while agitating; and

(b) filtering the resulting slurry.

In certain embodiments, the solvent is added over about 30 minutes, about 60 minutes, about 90 minutes, or even about 120 minutes, preferably over about 60 minutes.

In certain embodiments, the solvent is added while the temperature of the reaction is maintained in the range of about −10 to about 10° C., about −5 to about 5° C., or even about 0 to about 5° C.

In certain embodiments, the slurry is agitated for about 1 to about 24 hours. In certain preferred embodiments, the slurry is agitated for about 2 to about 10 hours, more preferably about 3 to about 5 hours, such as about 4 hours.

In certain embodiments, performing the reaction in the absence of pyridine may reduce the time required to dry a compound of formula (I).

In certain embodiments, addition of solvent to the reaction while agitating is done to accomplish precipitation of the compound of formula (I). Additionally, use of the solvent, e.g. acetone, may ultimately reduce the time required to dry a compound of formula (I) under reduced pressure by facilitating the removal of solvents and liquid impurities.

In certain embodiments, a compound of formula (I) may be dried in about 24 to about 48 hours. In certain embodiments, a compound of formula (I) may be dried under reduced pressure.

Another aspect of the invention relates to a method for purifying a compound of formula (I), e.g., following the above method, comprising

(a) adding an alcoholic and/or polar aprotic solvent, miscible with both ethanol and water that decreases the solubility of the compound of formula (I), preferably a dialkyl ketone such as acetone, to an aqueous or alcoholic solution of the compound while agitating; and

(b) filtering the resulting slurry.

In certain embodiments, the solvent is added over about 30 minutes, about 60 minutes, about 90 minutes, or even about 120 minutes, preferably over about 60 minutes.

In certain embodiments, the solvent is added while the temperature of the solution is maintained in the range of about −10 to about 10° C., about −5 to about 5° C., or even about 0 to about 5° C.

In certain embodiments, the slurry is agitated for about 1 to about 24 hours. In certain preferred embodiments, the slurry is agitated for about 2 to about 10 hours, more preferably about 3 to about 5 hours, such as about 4 hours.

As used herein, the term “agitate,” includes, but is not limited to, stirring (with a magnetic stir bar, a mechanical stirrer, or any other suitable stirring means) and shaking.

In certain embodiments, the method of purification further comprises

(a) preparing a solution of the compound of formula (I) in water;

(b) filtering the solution;

(c) reducing the amount of water (e.g., under reduced pressure and/or by azeotropic distillation);

(d) adding a water miscible, polar aprotic solvent that decreases the solubility of the compound (e.g., acetone, methyl isopropyl ketone, methyl ethyl ketone, or tetrahydrofuran), preferably a dialkyl ketone such as acetone, to form a slurry; and

(e) filtering the resulting slurry.

In certain embodiments, the compound of formula (I) is SGLU-1 as shown below

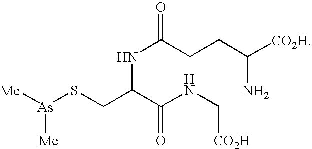

Another aspect of the invention relates to a method for the synthesis of a compound of formula (IV)

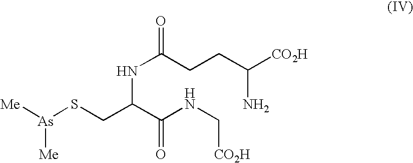

comprising reacting a compound having a structure of formula (V)

(Me)₂AsCl (V)

with a compound having a structure of formula (VI)

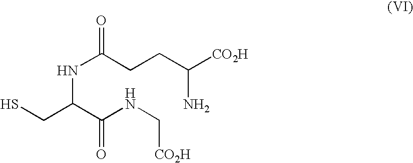

in an aqueous or alcoholic solvent and in the absence of pyridine to provide a compound of formula (IV).

In certain embodiments, the method further comprises

(a) adding an alcoholic and/or polar aprotic solvent, miscible with both ethanol and water that decreases the solubility of the compound of formula (I), preferably a dialkyl ketone such as acetone, to the reaction while agitating; and

(b) filtering the resulting slurry.

In certain embodiments, the solvent is added over about 30 minutes, about 60 minutes, about 90 minutes, or even about 120 minutes, preferably over about 60 minutes.

In certain embodiments, the solvent is added while the temperature of the reaction is maintained in the range of about −10 to about 10° C., about −5 to about 5° C., or even about 0 to about 5° C.

In certain embodiments, the slurry is agitated for about 1 to about 24 hours. In certain preferred embodiments, the slurry is agitated for about 2 to about 10 hours, more preferably about 3 to about 5 hours, such as about 4 hours.

Another aspect of the invention relates to a method for purifying a compound of formula (IV), e.g., following the above method, comprising

(a) adding an alcoholic and/or polar aprotic solvent, miscible with both ethanol and water that decreases the solubility of the compound of formula (I), preferably a dialkyl ketone such as acetone, to an aqueous or alcoholic solution of the compound while agitating; and

(b) filtering the resulting slurry.

In certain embodiments, the solvent is added over about 30 minutes, about 60 minutes, about 90 minutes, or even about 120 minutes, preferably over about 60 minutes.

In certain embodiments, the solvent is added while the temperature of the solution is maintained in the range of about −10 to about 10° C., about −5 to about 5° C., or even about 0 to about 5° C.

In certain embodiments, the slurry is agitated for about 1 to about 24 hours. In certain preferred embodiments, the slurry is agitated for about 2 to about 10 hours, more preferably about 3 to about 5 hours, such as about 4 hours.

In certain embodiments, the method of purification further comprises

(a) preparing a solution of the compound of formula (IV) in water;

(b) filtering the solution;

(c) reducing the amount of water (e.g., under reduced pressure and/or by azeotropic distillation);

(d) adding a water miscible, polar aprotic solvent that decreases the solubility of the compound (e.g., acetone, methyl isopropyl ketone, methyl ethyl ketone, or tetrahydrofuran), preferably a dialkyl ketone such as acetone, to form a slurry;

(e) filtering the resulting slurry.

Another aspect of the invention relates to a method for the crystallization of a compound of formula (I) or a compound of formula (IV) comprising

(a) dissolving the compound in an aqueous or alcoholic solvent;

(b) adding an alcoholic and/or polar aprotic solvent, miscible with both ethanol and water that decreases the solubility of the compound of formula (I), preferably a dialkyl ketone such as acetone, to the resulting solution while agitating; and

(b) filtering the resulting slurry.

In certain embodiments, the solvent is added over about 30 minutes, about 60 minutes, about 90 minutes, or even about 120 minutes, preferably over about 60 minutes.

In certain embodiments, the solvent is added while the temperature of the solution is maintained in the range of about −10 to about 10° C., about −5 to about 5° C., or even about 0 to about 5° C.

In certain embodiments, the slurry is agitated for about 1 to about 24 hours. In certain preferred embodiments, the slurry is agitated for about 2 to about 10 hours, more preferably about 3 to about 5 hours, such as about 4 hours.

In certain embodiments, such a method is performed such that the yield of the compound of formula (IV) is at least about 50%, about 60%, about 75%, about 80%, about 85%, about 90%, about 95% or even quantitative.

In certain embodiments, the compound of formula (IV) is at least about 97% pure as measured by HPLC and is free of pyridine. In certain preferred embodiments, the compound is at least about 99.5% pure.

Another aspect of the invention relates to a method for the synthesis of a compound of formula (II)

(R¹)(R²)AsCl (II)

wherein

R¹ and R² are independently C₁₋₂₀alkyl, preferably R¹ and R² are independently selected from methyl, ethyl, propyl, and isopropyl;

wherein a compound having the structure (VII)

(R¹)(R²)As(O)OH (VII)

is reduced with tin (II) chloride.

In certain embodiments, the reduction is performed as described in Example 1, wherein R¹ and R² are both methyl.

Previously, this conversion could be accomplished using hypophosphorus acid in concentrated hydrochloric acid; however, this resulted in the release of phosphine gas, a hazardous material. Reduction of a compound of formula (VII) with tin (II) chloride as described herein avoids generation of phosphine gas.

One aspect of the invention relates to a method for detecting the presence of a compound of Formula VIII

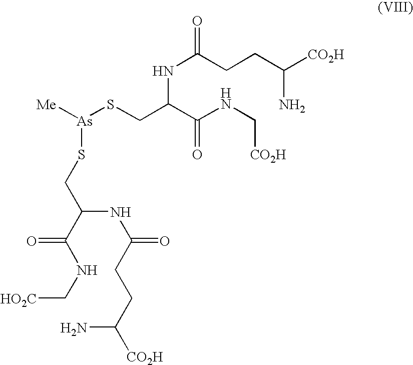

or salt thereof in a batch of SGLU-1, e.g., that has been manufactured as disclosed in WO 2007/027344, the disclosure of which is incorporated herein in its entirety.

As used herein, the term “batch” is meant to include the product of an SGLU-1 manufacturing process such that the amount of SGLU-1 produced is at least 1 kg, preferably at least 10 kg. Typically, a batch is at least 90% pure SGLU-1, although if the SGLU-1 has been mixed with other compounds, such as excipients, solvents, etc., prior to testing, then typically at least 90% of the arsenic-containing material in the sample is SGLU-1. In certain embodiments, such batches of SGLU-1 are substantially free of arsenic triglutathione, such that there is less than about 2%, less than about 1%, less than about 0.5%, or even less than about 0.25% triglutathione in the batch of SGLU-1. In certain embodiments, such batches of SGLU-1 are substantially free of biological contaminants, including, but not limited to, cells and proteins.

In certain embodiments, such a method may comprise detection using HPLC. In certain alternative embodiments, the method may comprise detection using mass spectrometry. In certain alternative embodiments, the method may comprise detection by NMR.

In certain embodiments, the invention relates to a method for assessing the purity of a sample of organic arsenical in which at least 90% of the organic arsenical in the sample is a compound of Formula IV

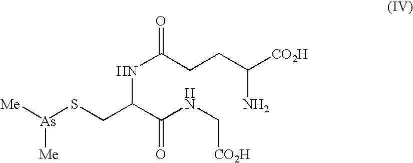

or a salt thereof, comprising detecting the presence of a compound of Formula VIII

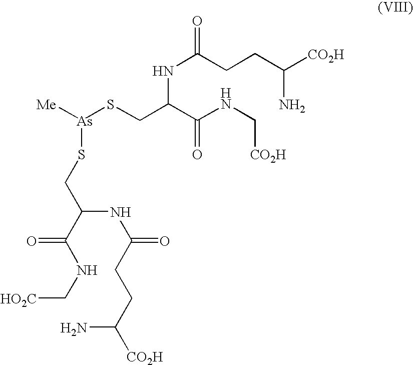

or a salt thereof in the sample. In certain such embodiments, detecting comprises analyzing the sample using HPLC. In certain alternative embodiments, detecting comprises analyzing the sample using mass spectrometry. In certain alternative embodiments, detecting comprises analyzing the sample using NMR.

In certain embodiments where the amount of the compound of Formula VIII detected is greater than about 5%, (total area as measured by HPLC), about 4%, about 3%, 2%, or even greater than about 1%, the sample may be purified to remove some or all of the compound of Formula VIII and then optionally retested. Such purification may be by any suitable means (e.g., recrystallization or HPLC purification). In circumstances where purification is not practical, such as when the sample is a multi-component pharmaceutical composition comprising the compound of Formula IV, the material from which the sample was taken may be discarded as unfit for human consumption.

In certain embodiments, the invention relates to a method for monitoring the presence of a compound of Formula VIII in a batch of SGLU-1, comprising detecting the amount of a compound of Formula VIII periodically over a time period of minutes, hours, days, weeks, or even years. In certain embodiments, the method comprises detecting the amount of a compound of Formula VIII at least once a day, once a week, once a month, or even at least once a year.

In certain embodiments where the method of assessing or monitoring comprises HPLC detection of a compound of Formula II, the HPLC analysis is performed at a temperature in the range of about 0 to about 20° C., preferably from about 4 to about 10° C. In certain such embodiments, the HPLC is performed at a temperature of about 4 to about 6° C.

In certain embodiments where the method of assessing or monitoring comprises HPLC detection of a compound of Formula VIII, the eluant may comprise a single uniform solution comprising at least one organic solvent. Such solutions may optionally further comprise water.

In certain embodiments, the eluant may comprise two or more solutions, each of which comprises at least one organic solvent. Such solutions may optionally further comprise water. In certain such embodiments where two or more solutions are used in changing proportions to vary the eluant along a gradient, the first solution may comprise an amine base and an organic acid. In certain such embodiments, the solution may comprise an amine base selected from triethylamine and diisopropylethylamine, preferably triethylamine. In certain such embodiments, the solution may comprise an organic acid, such as formic acid. In certain such embodiments, the solution may comprise triethylamine, formic acid, and water.

In certain embodiments where the first solution comprises water, formic acid, and triethylamine, the solution preferably comprises greater than about 95% water, greater than about 98%, or even greater than about 99% water. In certain such embodiments the solution comprises 99.85% water, 0.1% formic acid, and 0.05% triethylamine (v:v:v).

In certain embodiments where there are two solutions, the second solution may comprise an amine base and an organic acid. In certain such embodiments, the solution may comprise an amine base selected from triethylamine and diisopropylethylamine, preferably triethylamine. In certain such embodiments, the solution may comprise an organic acid, such as formic acid. In certain such embodiments, the solution may further comprise a water soluble organic solvent. In certain such embodiments, the water soluble organic solvent is acetonitrile. In certain such embodiments the solution comprises greater than about 98%, or even greater than about 99% acetonitrile. In certain embodiments, the solution comprises 99.85% acetonitrile, 0.1% formic acid, and 0.05% triethylamine (v:v:v).

One aspect of the invention relates to a method of manufacturing a pharmaceutical formulation of SGLU-1, comprising determining the amount of a compound of Formula VIII that is present and if the SGLU-1 comprises a compound of Formula VIII in an amount less than about 5% (total area as measured by HPLC), adding a pharmaceutically acceptable diluent, carrier, or excipient. In certain such embodiments, the SGLU-1 comprises a compound of Formula VIII in an amount less than about 4%, about 3%, 2%, or even less than about 1%. In certain embodiments, the pharmaceutical formulations are substantially free of arsenic triglutathione, such that there is less than about 2%, less than about 1%, less than about 0.5%, or even less than about 0.25% triglutathione in the batch of SGLU-1. In certain embodiments, the pharmaceutical formulations are substantially free of biological contaminants, including, but not limited to, cells and proteins.

In certain such embodiments, such pharmaceutical formulations may be used in the manufacture of oral dosage forms, including, but not limited to, capsules, tablets, pills, dragees, powders, granules, and the like.

Alternatively, such pharmaceutical formulations may be used in the manufacture of a solution suitable for intravenous administration.

One aspect of the invention relates to a method for detecting or monitoring the presence of a compound of Formula VIII or salt thereof in a pharmaceutical formulation, oral dosage form, or solution suitable for intravenous administration as described herein.

In certain embodiments, where the method is a method of detecting, such a method may comprise detection using HPLC. In certain alternative embodiments, the method may comprise detection using mass spectrometry. In certain alternative embodiments, the method may comprise detection by NMR.

In certain embodiments, where the method is a method for monitoring the presence of a compound of Formula VIII in a pharmaceutical formulation, oral dosage form, or solution suitable for intravenous administration, the method comprises detecting the amount of a compound of Formula VIII periodically over a time period of minutes, hours, days, weeks, or even years. In certain embodiments, the method comprises detecting the amount of a compound of Formula VIII at least once a day, once a week, once a month, or even at least once a year.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of an inhibitor(s) as an active ingredient. A composition may also be administered as a bolus, electuary, or paste.

In solid dosage forms for oral administration (capsules, tablets, pills, dragees, powders, granules, and the like), the active ingredient is mixed with one or more pharmaceutically acceptable carriers, such as sodium citrate or dicalcium phosphate, and/or any of the following: (1) fillers or extenders, such as starches, cyclodextrins, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise one or more inhibitors(s) in combination with one or more pharmaceutically acceptable sterile aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

Administration of the therapeutic compositions of the present invention to a patient will follow general protocols for the administration of chemotherapeutics, taking into account the toxicity, if any. It is expected that the treatment cycles would be repeated as necessary. It also is contemplated that various standard therapies or adjunct cancer therapies, as well as surgical intervention, may be applied in combination with the described arsenical agent.

Regardless of the route of administration selected, the inhibitor(s), which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

All publications and patents cited herein are hereby incorporated by reference in their entirety.

Those skilled in the art will recognize, or be able to ascertain using no more than routine experimentation, many equivalents to the specific embodiments of the invention described herein. Such equivalents are intended to be encompassed by the following claims.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art will, in light of the present disclosure, appreciate that many changes can be made in the specific embodiments which are disclosed and still obtain a like or similar result without departing from the spirit and scope of the invention.

### Example 1

### Preparation of Dimethylchloroarsine (DMCA)

A 3-neck round-bottom flask (500 mL) equipped with mechanical stirrer, inlet for nitrogen, thermometer, and an ice bath was charged with cacodylic acid (33 g, 0.23 mol) and conc. hydrochloric acid (67 mL). In a separate flask, a solution of SnCl₂.2H₂O (54 g, 0.239 mol) in conc. hydrochloric acid (10 mL) was prepared. The 5 nCl₂.2H₂O solution was added to the cacodylic acid in HCl solution under nitrogen while maintaining the temperature between 5° C. and 10° C. After the addition was complete, the ice bath was removed and the reaction mixture was stirred at ambient temperature for 1 h. The reaction mixture was transferred to a separatory funnel and the upper layer (organic) collected. The bottom layer was extracted with dichloromethane (DCM) (2×25 mL). The combined organic extract was washed with 1 N HCl (2×10 mL) and water (2×20 mL). The organic extract was dried over MgSO₄ and DCM was removed by rotary evaporation (bath temperature 80° C., under nitrogen, atmospheric pressure). The residue was further distilled under nitrogen. Two fractions of DMCA were collected. The first fraction contained some DCM and the second fraction was of suitable quality (8.5 g, 26% yield). The GC analysis confirmed the identity and purity of the product.

### Example 2

### Preparation of S-Dimethylarsinoglutathione (SGLU-1)

A suspension of glutathione (18 g, 59 mmol) in a mixture of water/ethanol 1:1 v/v (180 mL) was cooled below 5° C. and under an inert atmosphere treated with triethylamine (10 mL, 74 mmol) in one portion. The mixture was cooled to 0-5° C. and DMCA (11 g, 78.6 mmol) was added dropwise over a period of 10 min, while maintaining the temperature below 5° C. The reaction mixture was stirred at 0-5° C. for 4 h, and the resulting solids were isolated by filtration. The product was washed with ethanol (2×50 mL) and acetone (2×50 mL) and dried in vacuum at RT overnight, to give 11 g (46%) of SGLU-1. HPLC purity was 97.6% by area (average of 3 injections), Anal. Calcd. for C₁₂H₂₂AsN₃O₆S: C, 35.04; H, 5.39; N, 10.12, S, 7.8. Found: C, 34.92; H, 5.31; N, 10.27, S, 7.68. ¹H and ¹³C-NMR were consistent with the structure. The filtrate was diluted with acetone (150 mL) and placed in a refrigerator for 2 days. An additional 5.1 g (21%) of SGLU-1 was isolated as the second crop, HPLC purity was 97.7% by area (average of 3 injections).

### Example 3

### Preparation of S-Dimethylarsinoglutathione (SGLU-1)

In a 3 L three-neck flask equipped with a mechanic stirrer, dropping funnel and thermometer under an inert atmosphere was prepared a suspension of glutathione (114.5 g, 0.37 mol) in a 1:1 (v/v) mixture of water/ethanol (1140 mL) and cooled to below 5° C. The mixture was treated slowly (over 15 min) with triethylamine (63.6 mL, 0.46 mol) while maintaining the temperature below 20° C. The mixture was cooled to 4° C. and stirred for 15 min and then the traces of undissolved material removed by filtration. The filtrate was transferred in a clean 3 L three-neck flask equipped with a mechanic stirrer, dropping funnel, nitrogen inlet, and thermometer and DMCA (70 g, 0.49 mol) (lot # 543-07-01-44) was added slowly while maintaining the temperature at 3-4° C. The reaction mixture was stirred at 1-4° C. for 4 h, and acetone (1.2 L) was added over a period of 1 h. The mixture was stirred for 90 min between 2 and 3° C. and the resulting solid was isolated by filtration. The product was washed with ethanol (2×250 mL) and acetone (2×250 mL) and the wet solids were suspended in ethanol 200 Proof (2000 mL). The product was isolated by filtration, washed with ethanol (2×250 mL) and acetone (2×250 mL) and dried in vacuum for 2 days at RT to give 115 g (75%) of SGLU-1, HPLC purity >99.5% (in process testing).

### Example 4

### Purification of SGLU-1

A suitable reactor was charged with SGLU-1 (6.0 kg) and water (72 kg). The slurry was heated to 30 to 40° C. until a solution formed, and the resulting solution was pumped through a 1.2 μm inline filter to remove any particulate matter present in the solution. The clarified solution was then concentrated under reduced pressure using a rotary evaporator. The water bath was maintained at not more than 40° C. When ca. 85% of the total solution volume of water was removed, ethanol (30 L, 200 proof, USP) was added to the concentrate, and distillation was continued at 40° C. to azeotropically remove the remaining water until a slurry began to form. The slurry was then diluted with acetone (24 L) at which point a solution formed. The solvents were removed under reduced pressure until a slurry formed. The resulting slurry was transferred to a suitable reactor and was diluted with acetone (6 L).

The mix was chilled to 0 to 5° C. and was allowed to stand for one hour. The SGLU-1 was then filtered and washed with acetone (3 L) and dried under reduced pressure to provide SGLU-1 (5.1 kg, 85% yield).

### Example 5

### HPLC Determination of Purity of ZIO-101

The following conditions may be used to determine the area % of SGLU-1 in order to determine the purity of a sample:

Column: C8 or C18

Mobile Phase: A: pH 2-5; B acetonitrile

Injection volume: 1 to 10 μl

Temp: 0-25° C.

Wavelength: 210-250 nm

Sample prep: 10-30 mg

In certain embodiments, the following conditions may be used:

| Column | Phenomenex Gemini C18 column, 150 × | | |
|---|---|---|---|
| | 4.6 mm, 3μ, 110 Å | | |
| Mobile phase: | Time (min) | A (% v/v) | B (% v/v) |
| A = 25 mM ammonium formate pH 3.02 | 0 | 95 | 5 |
| B = acetonitrile | 6 | 95 | 5 |
| | 20 | 65 | 35 |
| | 21 | 65 | 35 |
| | 21.1 | 95 | 5 |
| | 30 | 95 | 5 |

| | |
|---|---|
| Injection volume | 5.0 μL |
| Temperature | Column and autosampler at 5° C. |
| Wavelength | 230 nm |
| Temperature | 5° C. |
| Sample preparation | 25 mg sample was dissolved in cooled (5° C.) |
| | mobile phase A plus 0.05% TFA in a 25 mL |
| | volumetric flask and mixed, q.s. |

### Example 6

The compound of Formula VIII was separated from SGLU-1 using the following

HPLC conditions:

HPLC Conditions:

| | |
|---|---|
| Mobile Phase A | Water:Formic Acid:Triethylamine |
| | 99.85:0.1:0.05 (v:v:v) |
| Mobile Phase B | Acetonitrile:Formic Acid:Triethylamine |
| | 99.85:0.1:0.05 (v:v:v) |
| Flow | 1.0 mL/min |
| Post Column Pre Probe Split | ⅓ Flow Rate |
| Column Temperature | 6° C. |
| Autosampler | 5° C. |
| Injection Volume | 1 μL or 20 μL |
| Run Time | 30 minutes |

A gradient was run according to the following parameters:

| Time (minutes) | % Mobile Phase A | % Mobile Phase B |
|---|---|---|
| 0.0 | 95 | 5 |
| 20.0 | 85 | 15 |
| 21.1 | 95 | 5 |
| 30.0 | 95 | 5 |

Chromatographic separation showed that the compound of Formula VIII was present in an amount of 1.5 to 2.0% of the total peak area.

Mass spectrometry of the isolated peak for the compound of Formula VIII was performed in order to confirm the expected mass. The synthesis of the compound of Formula VIII has been reported in the literature and the available MS data (JAAS 2004; 19:183; J. Biol. Chem. 275(43):33404), are consistent with the data observed herein.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also recognize that all combinations of embodiments described herein are within the scope of the invention.

We claim:

1. A method for the synthesis of a compound of formula (I) 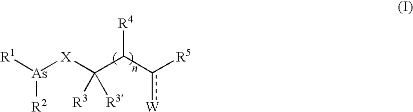
wherein
X is S or Se;
W is O, S, or (R)(R), where each occurrence of R is independently H or a C₁₋₂alkyl;
n is 0 or 1;
R¹ and R² are independently C₁₋₂₀alkyl;
R³ is —H or C₀₋₆alkyl-COOR⁶;
R^{3′} is H, amino, cyano, halogen, aryl, aralkyl, heteroaryl, heteroaralkyl, carboxyl, C₁₋₁₀alkyl, C₁₋₁₀alkenyl, or C₁₋₁₀alkynyl;
R⁴ is —OH, —H, —CH₃, —OC(O)C₁₋₁₀aralkyl, —OC(O)C₁₋₁₀alkyl, —OC(O)aryl, or a glutamine;
R⁵ is —OH, cyano, C₁₋₁₀alkoxy, amino, O-aralkyl, —OC(O)C₁₋₁₀aralkyl, —OC(O)C₁₋₁₀alkyl, —OC(O)aryl, or a glycine substituent; and
R⁶ is H or C₁₋₁₀alkyl,
comprising reacting a compound having a structure of formula (II)
(R¹)(R²)AsCl (II)
with a compound having a structure of formula (III) 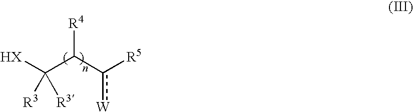
in an aqueous or alcoholic solvent and in the absence of pyridine to provide a compound of formula (I).

2. A method of claim 1, wherein the compound of formula (III) has a structure

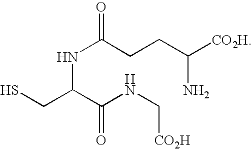

3. A method of claim 1 or 2, wherein the reaction is conducted in the presence of a non-aromatic amine base.

4. A method of claim 3, wherein the non-aromatic amine base is triethylamine.

5. A method of any one of claims 1 to 4, wherein the solvent system comprises ethanol and water.

6. A method of claim 5, wherein the ratio of water to ethanol (v/v) is between about 2:1 and about 1:2.

7. A method of claim 6, wherein the ratio of water to ethanol (v/v) is about 1:1.

8. A method for purifying a compound of formula (I), 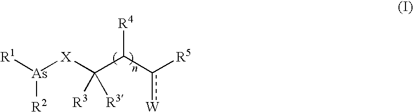
wherein
X is S or Se;
W is O, S, or (R)(R), where each occurrence of R is independently H or a C₁₋₂alkyl;
n is 0 or 1;
R¹ and R² are independently C₁₋₂₀alkyl;
R³ is —H or C₀₋₆alkyl-COOR⁶;
R^{3′} is H, amino, cyano, halogen, aryl, aralkyl, heteroaryl, heteroaralkyl, carboxyl, C₁₋₁₀alkyl, C₁₋₁₀alkenyl, or C₁₋₁₀alkynyl;
R⁴ is —OH, —H, —CH₃, —OC(O)C₁₋₁₀aralkyl, —OC(O)C₁₋₁₀alkyl, —OC(O)aryl, or a glutamine;
R⁵ is —OH, cyano, C₁₋₁₀alkoxy, amino, O-aralkyl, —OC(O)C₁₋₁₀aralkyl, —OC(O)C₁₋₁₀alkyl, —OC(O)aryl, or a glycine substituent; and
R⁶ is H or C₁₋₁₀alkyl
comprising
(a) adding an alcoholic or polar aprotic solvent, miscible with both ethanol and water that decreases the solubility of the compound of formula (I) to an aqueous or alcoholic solution of the compound while agitating; and
(b) filtering the resulting slurry.

9. A method of claim 8, wherein the compound of formula (I) is 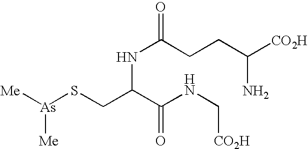
or a pharmaceutically acceptable salt thereof.

10. A method of claim 9, wherein the alcoholic or aprotic solvent is acetone.

11. A method of claim 8 or 9, further comprising
(a) preparing a solution of the compound of formula (I) in water;
(b) filtering the solution;
(c) reducing the amount of water;
(d) adding a water miscible, polar aprotic solvent that decreases the solubility of the compound to form a slurry; and
(e) filtering the resulting slurry.

12. A method for the synthesis of a compound having a structure of formula (II)
(R¹)(R²)AsCl (II)
wherein
R¹ and R² are independently C₁₋₂₀alkyl, preferably independently selected from methyl, ethyl, propyl, and isopropyl;
comprising reducing a compound having the structure (VII)
(R¹)(R²)As(O)OH (VII)
with tin (II) chloride.

13. A method of claim 12, wherein R¹ and R² are independently selected from methyl, ethyl, propyl, and isopropyl.

14. A method of claim 13, wherein R¹ and R² are both methyl.

15. A compound of formula (IV) 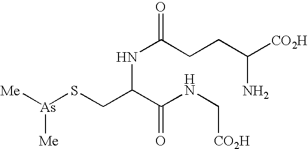
wherein the compound at least 97% pure as measured by HPLC; and
the compound is free of pyridine.

16. A method for assessing the purity of a sample of organic arsenical in which at least 90% of the organic arsenical in the sample is a compound of Formula IV 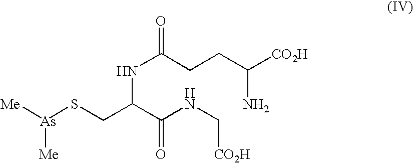
or a salt thereof, comprising detecting the presence of a compound of Formula VIII 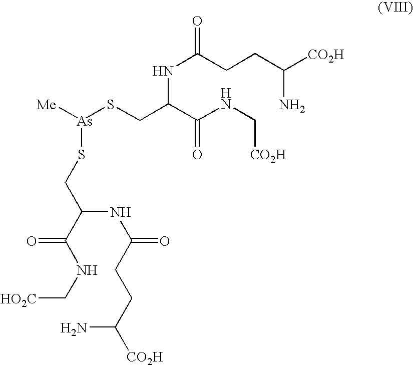
or a salt thereof in the sample.

17. A method of claim 16, wherein detecting comprises analyzing the sample using HPLC.

18. A method of claim 16, wherein detecting comprises analyzing the sample using mass spectrometry.

19. A method of claim 16, wherein detecting comprises analyzing the sample using NMR.

20. A method for monitoring a sample for the presence of a compound of Formula VIII 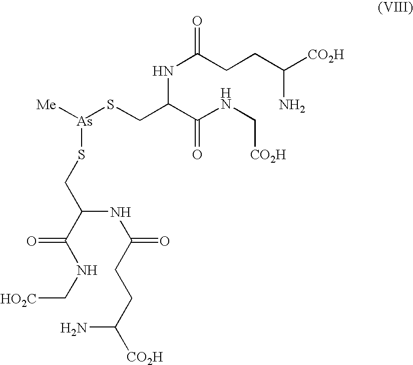
or a salt thereof, comprising detecting the amount of a compound of Formula VIII or salt thereof in the sample according to any one of claims 1 to 4 once a week for a period of a month.

21. A method of manufacturing a pharmaceutical formulation of a compound of Formula IV 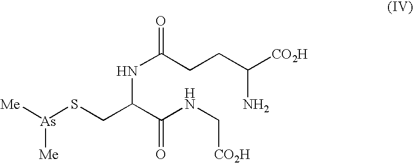
or a salt thereof, comprising determining the amount of a compound of Formula VIII 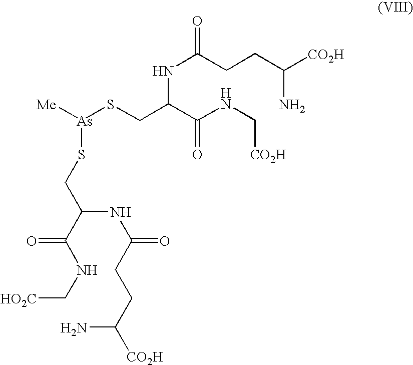
or salt thereof that is present in a sample of the compound of Formula IV and if the amount of the compound of Formula VIII is less than about 5% (w/w) relative to the compound of Formula IV, adding a pharmaceutically acceptable diluent, carrier, or excipient.

22. A method of claim 21, wherein the compound of Formula VIII is present in an amount less than about 2% relative to the compound of Formula IV.

23. A method of claim 22, wherein the compound of Formula VIII is present in an amount less than about 1% relative to the compound of Formula IV.

24. A pharmaceutical formulation manufactured according to the method of any one of claims 21 to 23.

25. An oral dosage form, wherein the oral dosage form comprises a pharmaceutical composition of claim 24.
